(19) United States
(12) Patent Application Publication
(10) Pub. No.: US 2009/0176717 A1
(43) Pub. Date: Jul. 9, 2009

(54) GALACTOSIDES AND THIODIGALACTOSIDES AS INHIBITORS OF PA-IL LECTIN FROM PSEUDOMONAS

(75) Inventor: John L. Magnani, Gaithersburg, MD (US)

Correspondence Address: SEED INTELLECTUAL PROPERTY LAW GROUP PLLC, 701 FIFTH AVE, SUITE 5400, SEATTLE, WA, 98104, US

(73) Assignee: GlycoMimetics, Inc., Gaithersburg, MD (US)

(21) Appl. No.: 12/302,092

(22) PCT Filed: May 30, 2007
(86) PCT No.: PCT/US2007/012867

§ 371 Date: Nov. 24, 2008

Related U.S. Application Data
(60) Provisional application No. 60/810,189, filed on Jun. 1, 2006.

(51) Int. Cl. A61K 31/7016; G01N 33/53; C12N 11/00

(52) U.S. Cl. 514/24; 435/7.1; 435/174

(57) ABSTRACT

Compositions and methods are provided related to Pseudomonas bacteria. The compositions and methods may be used for diagnosis and therapy of medical conditions involving infection with Pseudomonas bacteria. Such infections include Pseudomonas aeruginosa in the lungs of patients with cystic fibrosis. A compound useful in the present methods may be used in combination with a therapeutic agent or may be linked to a therapeutic agent. Pseudomonas bacteria may be inhibited by blocking colonization, inhibiting virulence factors, arresting growth or killing the bacteria.

### BACKGROUND OF THE INVENTION

1. Field of the Invention

The present invention relates generally to compositions and methods for the diagnosis and therapy of diseases in warm-blooded animals (e.g., in humans) involving infections with and colonization by Pseudomonas bacteria, including Pseudomonas aeruginosa in the lungs of patients with cystic fibrosis. The invention relates more particularly to the use of one or more compounds selective for binding PA-IL lectin of Pseudomonas bacteria. These compounds are useful for diagnosis and/or therapeutic intervention of the colonization of Pseudomonas bacteria, or may be linked to an agent(s) to target and effectively arrest or kill Pseudomonas bacteria.

2. Description of the Related Art

Pseudomonas infections occur in a variety of medical conditions and can be life threatening. Pseudomonas is an opportunistic bacterium. Examples of individuals at risk include cystic fibrosis patients, burn patients, surgery patients, and patients on ventilators. Cystic fibrosis is described below as a representative example of a medical condition which can involve infection with Pseudomonas bacteria.

Cystic Fibrosis (CF) is the most common lethal genetic disease among the Caucasian population. CF is caused by mutations in the gene encoding the cystic fibrosis transmembrane conductance regulator (CFTR), which acts as a chloride channel. The genetic mutations of CFTR which alter ion movements also affect the N-glycosylation of CFTR as well as other cell surface molecules. All of the exocrine glands of the patients are affected; however, the lungs are the primary site of morbidity and mortality. The general change in glycosylation is associated with an increase in infectivity by Pseudomonas aeruginosa. The salivary and respiratory mucins from CF patients also contain altered glycosylation patterns.

The major cause of morbidity and mortality in CF patients is chronic lung colonization by the bacterium, Pseudomonas aeruginosa, which results in pronounced lung infection with a robust neutrophilic inflammatory response leading to destruction of the lungs and death. Colonization by P. aeruginosa initiates during the sessile phase of the bacteria in which virulence factors are secreted in concert. Two virulence factors that bind carbohydrates are lectins. These lectins, known as PA-IL and PA-IIL, bind these oligosaccharide structures with high affinity and represent a potential molecular target to block bacterial colonization. Patients that are never fully colonized by the bacteria maintain an excellent long-term prognosis. Due to the difficulties in the current approaches in the art for prevention of colonization in an individual by Pseudomonas bacteria, there is a need for improved compositions and methods.

### BRIEF SUMMARY OF THE INVENTION

Briefly stated, this invention provides compositions and methods for utilizing PA-IL lectin expressed by Pseudomonas bacteria for the detection of Pseudomonas bacteria and the diagnosis and therapy of disease involving Pseudomonas bacteria, including human disease. For example, a compound in a composition of the present invention that surprisingly has high affinity binding to PA-IL lectin from P. aeruginosa will have a beneficial therapeutic effect on CF patients. In addition, the compound may be administered in combination with another inhibitor of Pseudomonas bacteria. Furthermore, the compound may be administered in combination therapy with an antibiotic or may be conjugated, for example, with an antibiotic to increase the efficacy and lower the dose, thereby avoiding dose-related deleterious side effects of the antibiotic. Given that these binding sites are crucial for the colonization and pathogenicity of the bacterium, mutations in this target to become resistant to this therapy should result in non-pathogenic forms of the bacteria.

One embodiment of the present invention provides a composition comprising a pharmaceutically acceptable carrier or diluent in combination with a compound or physiologically acceptable salt thereof, the compound with the formula:

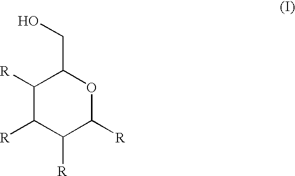

wherein each R is independently selected from OH, NHAc, alkyl, O-alkyl, S-alkyl, cycloalkyl, O-cycloalkyl, S-cycloalkyl, heterocycle, O-heterocycle, S-heterocycle, aryl, O-aryl, S-aryl, heteroaryl, O-heteroaryl, S-heteroaryl, and

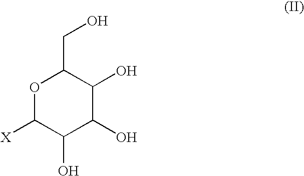

where X is S, O or CH₂, and with the bond of an R to the X of formula (II) where an R is selected from formula (II); and

with the proviso that the R of formula (I) are not all OH.

In another embodiment, the present invention provides a composition comprising another inhibitor of Pseudomonas bacteria in combination with a compound as set forth above.

A compound or salt thereof of a composition of the present invention may be in combination with a pharmaceutically acceptable carrier or diluent.

In another embodiment, the compound of a composition of the present invention is with the formula:

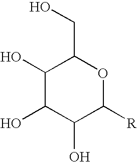

where R is as defined above.

In another embodiment, the compound of a composition of the present invention is with the formula:

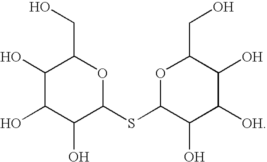

In another embodiment, the compound of a composition of the present invention is with the formula:

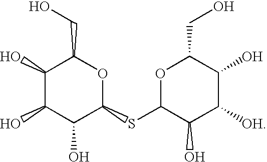

In another embodiment, a composition of the present invention further includes a therapeutic agent for Pseudomonas bacteria therapy.

In another embodiment, a compound of a composition of the present invention is attached to a therapeutic agent for Pseudomonas bacteria therapy.

Another embodiment of the present invention provides a conjugate comprising a compound or physiologically acceptable salt thereof joined covalently to a therapeutic agent for Pseudomonas bacteria therapy, the compound with the formula:

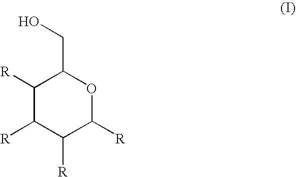

wherein each R is independently selected from OH, NHAc, alkyl, O-alkyl, S-alkyl, cycloalkyl, O-cycloalkyl, S-cycloalkyl, heterocycle, O-heterocycle, S-heterocycle, aryl, O-aryl, S-aryl, heteroaryl, O-heteroaryl, S-heteroaryl, and

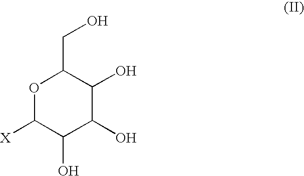

where X is S, O or CH₂, and with the bond of an R to the X of formula (II) where an R is selected from formula (I); and

with the proviso that the R of formula (I) are not all OH.

In another embodiment, the compound of a conjugate of the present invention is with the formula:

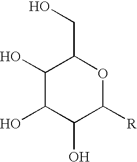

where R is as defined above.

In another embodiment, the compound of a conjugate of the present invention is with the formula:

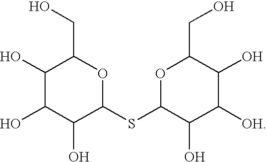

In another embodiment, the compound of a conjugate of the present invention is with the formula:

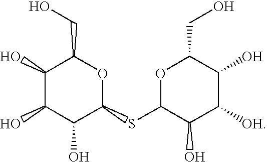

Another embodiment of the present invention provides a method of inhibiting Pseudomonas bacteria infection in a warm-blooded animal comprising administering to the animal in an amount effective to inhibit PA-IL lectin of the bacteria a composition comprising a composition of the present invention.

In another embodiment, the present invention provides a method of detecting Pseudomonas bacteria comprising contacting a sample with a diagnostic agent linked to a compound as set forth above, under conditions sufficient for the compound to bind to the bacteria or PA-IL lectin product if present in the sample; and detecting the agent present in the sample, wherein the presence of agent in the sample is indicative of the presence of Pseudomonas bacteria.

In another embodiment, the present invention provides a method of immobilizing Pseudomonas bacteria on a solid support comprising contacting, under conditions sufficient for binding, a sample containing Pseudomonas bacteria with a compound comprising a compound as set forth above that is immobilized on a solid support; and separating the sample from the solid support.

In other embodiments, the methods described herein are used specifically where the Pseudomonas bacteria are Pseudomonas aeruginosa.

In other embodiments, the compounds, compositions and conjugates described herein may be used for first and second medical uses, e.g., in the preparation of a medicament for the inhibition of Pseudomonas bacteria, including Pseudomonas aeruginosa.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 shows the determination of IC₅₀ value for thiodigalactoside for inhibition of PA-IL.

FIG. 2 shows the inhibitory activity of galactose mimics for PA-IL Lectin relative to Galactose (rIC50).

FIG. 3 shows the inhibitory activity of a galactose mimic for PA-IL Lectin relative to Galactose (rIC50).

### DETAILED DESCRIPTION OF THE INVENTION

Prior to setting forth embodiments of the present invention, it may be helpful to an understanding thereof to set forth definitions of certain terms to be used hereinafter.

“Alkyl” refers to straight- or branched-chain hydrocarbons having from 1 to 20 carbon atoms in the chain. The alkyl group may be substituted or unsubstituted on the chain, and may include one or more carbon-carbon double or triple bonds.

“Cycloalkyl” refers to a cyclic alkyl group that contains between 3 and 8 carbon atoms and has a single cyclic ring. The cycloalkyl ring may be substituted or unsubstituted, and may be preceded by one or more CH₂ groups.

“Heterocycle” refers to a monocyclic or fused ring (i.e., rings which share an adjacent pair of atoms) group having in the ring(s) one or more heteroatoms, preferably selected from nitrogen, oxygen and sulfur. The ring(s) may also have one or more double bonds. However, the ring(s) are not aromatic. The ring(s) may be substituted or unsubstituted, and may be preceded by one or more CH₂ groups.

“Aryl” refers to an unsaturated aromatic carbocyclic group of 6 to 14 carbon atoms having a single ring or multiple condensed rings. The aryl group may be substituted or unsubstituted, and may be preceded by one or more CH₂ groups.

“Heteroaryl” refers to a monocyclic or fused ring aryl group having in the ring(s) one or more heteroatoms, preferably selected from nitrogen, oxygen and sulfur. The heteroaryl group may be substituted or unsubstituted, and may be preceded by one or more CH₂ groups.

As noted above, the present invention provides compositions and conjugates that bind Pseudomonas bacteria (e.g., P. aeruginosa) and may be used in the diagnosis and therapy of disease.

### Compounds and Compositions

The compositions of the present invention possess a compound (including physiologically acceptable salts thereof) that unexpectedly has high affinity for PA-IL lectin from Pseudomonas bacteria. Embodiments of a compound in a composition of the present invention are depicted with the formula:

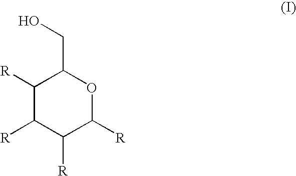

wherein each R is independently selected from OH, NHAc, alkyl, O-alkyl, S-alkyl, cycloalkyl, O-cycloalkyl, S-cycloalkyl, heterocycle, O-heterocycle, S-heterocycle, aryl, O-aryl, S-aryl, heteroaryl, O-heteroaryl, S-heteroaryl, and

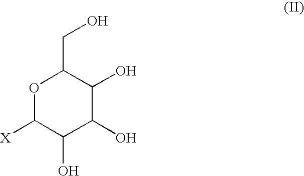

where X is S, O or CH₂, and with the bond of an R to the X of formula (II) where an R is selected from formula (II); and

with the proviso that the R of formula (I) are not all OH.

In an embodiment, the compound of a composition of the present invention is with the formula:

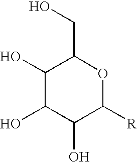

where R is as defined above.

In an embodiment, the compound of a composition of the present invention is with the formula:

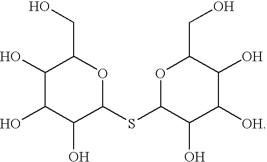

In an embodiment, the compound of a composition of the present invention is with the formula:

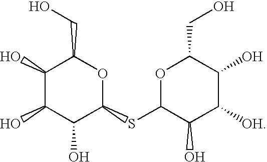

In compound (I) above, there are four R substituents. Options for R substituents are set forth above, for example, OH and NHAc. Each R is independently selected, with the exception that the R of formula (I) are not all OH. In one embodiment, three of the R are OH (hydroxyl groups). In other embodiments, any three of the four R substituents are independently selected from OH and NHAc and the fourth R is not OH or NHAc. (For example, one of the R substituents is NHAc, two are OH and the fourth is not OH or NHAc.) An R may be also selected from an alkyl group (as defined herein). Examples of an alkyl substituent include methyl, ethyl, n-propyl, i-propyl, n-butyl, t-butyl, propenyl and butenyl. Where an alkyl group is a straight-chain hydrocarbon without substitution or double or triple bonds, it will be CH₃ or (CH₂)ₙCH₃ where n is 1-20. An alkyl group may also be in the form of O-alkyl or S-alkyl. An R may be also selected from a substituent with the formula:

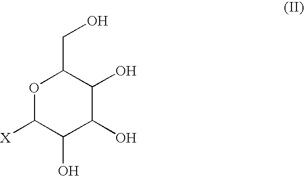

where X is S, O or CH₂.

Where R is selected from the substituent with formula (II), the bond on the R joins compound (I) to substituent (II) by X of formula (II). An R may be also selected from a cyclic substituent such as cycloalkyl, heterocycle, aryl, and heteroaryl (all as defined herein). These substituents may also be in the form O-cycloalkyl, S-cycloalkyl, O-heterocycle, S-heterocycle, O-aryl, S-aryl, O-heteroaryl or S-heteroaryl. Examples of a cycloalkyl substituent include cyclopropyl, cyclopentyl, cyclohexyl, and cyclooctyl. Examples of a heterocycle substituent include piperidine, piperazine and morpholine. Examples of an aryl substituent include phenyl, naphthyl and anthryl. Examples of a heteroaryl substituent include pyrrole, furan, thiophene, imidazole, oxazole, thiazole, pyrazole, pyridine, pyrimidine, quinoline, isoquinoline, purine and carbazole.

In another embodiment, there is only one R substituent and the compound formula is depicted above where the single R is attached to a ring carbon bonded to the ring oxygen. The possible substituents for this R are the same as described above in the context of the R of compound (I).

Where the R attached to a ring carbon bonded to the ring oxygen is with the formula:

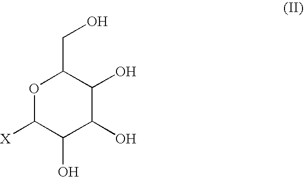

then the two rings, i.e., formula (I) and formula (II), are joined via the X. Where X is S and with the appropriate stereochemistry (depicted above as [symbol] or [symbol] ) for the attachment of each of the hydroxyl groups to both rings, the compound formed is thiodigalactoside.

A compound of a composition of the present invention may be synthesized using methodology and protocols known to one of skill in the art. Certain such compounds are also commercially available. Sources include Sigma Chemical Co. (St. Louis, Mo.) and Toronto Research Chemicals, Inc. (North York, Ontario, Canada).

An embodiment of a composition of the present invention comprises a compound or physiologically acceptable salt thereof as set forth above in combination with a pharmaceutically acceptable carrier or diluent. In addition to a pharmaceutically acceptable carrier or diluent, or as an alternative thereto, a compound or physiologically acceptable salt thereof as set forth above is combined with another inhibitor of Pseudomonas bacteria in embodiments of a composition of the present invention. As used herein, the term “another inhibitor” means one or more inhibitors of Pseudomonas bacteria, and may be inhibitors of PA-IL lectin that are other than the compounds set forth above in the compositions of the present invention. An example of another inhibitor of Pseudomonas bacteria is polyethylene glycol 15-20 (Wu et al., Gastroenterology 126:488-498, 2004).

For certain embodiments, it may be beneficial to also, or alternatively, link a diagnostic or therapeutic agent, such as a drug to a compound, to form a conjugate where the linkage is covalent. As used herein, the term “therapeutic agent” refers to any bioactive agent intended for administration to a warm-blooded animal (e.g., a mammal such as a human) to prevent or treat a disease or other undesirable condition, or to enhance the success of therapies against diseases or conditions, associated with Pseudomonas bacteria (such as Pseudomonas aeruginosa). Therapeutic agents include antibiotics, hormones, growth factors, proteins, peptides, genes, non-viral vectors and other compounds.

### Formulations

Compounds as described herein may be present within a pharmaceutical composition. A pharmaceutical composition comprises a compound in combination with one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate. Compositions of the present invention may be formulated for any appropriate manner of administration, including for example, aerosol, topical, parenteral, oral, nasal, intravenous, intracranial, intraperitoneal, subcutaneous, or intramuscular administration.

A pharmaceutical composition may also, or alternatively, contain one or more active agents, such as drugs (e.g., antibiotics), which may be attached to a compound or may be included free within the composition. The attachment of an agent to a compound may be covalent or noncovalent. An example of an active agent is tobramycin. Tobramycin alone has typically been administered intravenously or by inhalation.

The compositions described herein may be administered as part of a sustained release formulation (i.e., a formulation such as a capsule or sponge that effects a slow release of modulating agent following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of modulating agent release. The amount of compound contained within a sustained release formulation depends upon the site of implantation, the rate and expected duration of release and the nature of the condition to be treated or prevented.

Compounds are generally present within a pharmaceutical composition in a therapeutically effective amount. A therapeutically effective amount is an amount that results in a discernible patient benefit, such as a measured or observed response of a condition associated with Pseudomonas infection.

### Methods of Use

In general, compounds described herein may be used for achieving diagnostic and/or therapeutic results in disease (e.g., human disease) involving infection by Pseudomonas (e.g., P. aeruginosa) bacteria. Such diagnostic and/or therapeutic results may be achieved in vitro and/or in vivo in an animal, preferably in a mammal such as a human, provided that Pseudomonas (e.g., P. aeruginosa) or its lectin products are ultimately contacted with a compound, in an amount and for a time sufficient to achieve a discernable diagnostic or therapeutic result. In the context of this invention, a therapeutic result would relate, for example, to the prevention of lung infections. In some conditions, therapeutic results would be associated with the inhibiting of Pseudomonas (such as P. aeruginosa) or its products (where inhibiting includes, for example, arresting the growth of or killing the bacteria or preventing colonization by the bacteria). As used herein, therapy or therapeutic results includes treatment or prevention.

Compounds in compositions of the present invention may be administered in a manner appropriate to the disease to be treated or prevented. Appropriate dosages and a suitable duration and frequency of administration may be determined by such factors as the condition of the patient, the type and severity of the patient's disease and the method of administration. In general, an appropriate dosage and treatment regimen provides the compound(s) in an amount sufficient to provide treatment and/or prophylactic benefit. Within particularly preferred embodiments of the invention, a compound may be administered in a composition of the present invention at a dosage ranging from 0.001 to 1000 mg/kg body weight (more typically 0.01 to 1000 mg/kg), on a regimen of single or multiple daily doses. Appropriate dosages may generally be determined using experimental models and/or clinical trials. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Compounds as set forth above may also be used to target substances to Pseudomonas bacteria, e.g., P. aeruginosa. Such substances include therapeutic agents and diagnostic agents. Therapeutic agents may be a molecule, virus, viral component, cell, cell component or any other substance that can be demonstrated to modify the properties of a target cell so as to provide a benefit for treating or preventing a disorder or regulating the physiology of a patient. A therapeutic agent may also be a drug, or a prodrug that generates an agent having a biological activity in vivo. Molecules that may be therapeutic agents may be, for example, polypeptides, amino acids, nucleic acids, polynucleotides, nucleosides, steroids, polysaccharides or inorganic compounds. Such molecules may function in any of a variety of ways, including as enzymes, enzyme inhibitors, hormones, receptors, antisense oligonucleotides, catalytic polynucleotides, anti-viral agents, anti-tumor agents, anti-bacterial agents, immunomodulating agents and cytotoxic agents (e.g., radionuclides such as iodine, bromine, lead, rhenium, homium, palladium or copper). Diagnostic agents include imaging agents such as metals and radioactive agents (e.g., gallium, technetium, indium, strontium, iodine, barium, bromine and phosphorus-containing compounds), contrast agents, dyes (e.g., fluorescent dyes and chromophores) and enzymes that catalyze a calorimetric or fluorometric reaction. In general, therapeutic and diagnostic agents may be attached to a compound using a variety of techniques such as those that are well known in the art. For targeting purposes, a compound may be administered to a patient as described herein.

Compounds as set forth above may also be used in vitro, e.g., within a variety of well known cell culture and cell separation methods. For example, a compound may be immobilized on a solid support (such as linked to the interior surface of a tissue culture plate or other cell culture support) for use in immobilizing Pseudomonas bacteria or their products for screens, assays and growth in culture. Such linkage may be performed by any suitable technique, such as standard techniques known in the art. Compounds may also be used to facilitate cell identification and sorting in vitro, permitting the selection of such bacterial cells. Preferably, the compound(s) for use in such methods is linked to a diagnostic agent which is a detectable marker. Suitable markers are well known in the art and include radionuclides, luminescent groups, fluorescent groups, enzymes, dyes, constant immunoglobulin domains and biotin. Within one preferred embodiment, a compound linked to a fluorescent marker, such as fluorescein, is contacted with the cells, which are then analyzed by fluorescence activated cell sorting (FACS).

Such in vitro methods generally comprise contacting a sample (e.g., a biological preparation) with a compound, and detecting the compound in the sample. If desired, one or more wash steps may be added to a method. For example, subsequent to contacting a sample with a compound but prior to detection of the compound, the sample may be washed (i.e., contacted with a fluid and then removal of the fluid in order to remove unbound compound). Alternatively, or in addition, a wash step may be added during the detection process. For example, if a compound possesses a marker (a diagnostic agent) that can bind to a substance that is detectable, it may be desirable to wash the sample subsequent to contacting the sample with a detectable substance, but prior to the detection. As used herein, the phrase “detecting the compound (or agent) in the sample” includes detecting the compound (or agent) while it is bound to the sample, or detecting the compound (or agent) which was bound to the sample but after it has been separated from the sample.

The following Examples are offered by way of illustration and not by way of limitation.

### EXAMPLES

### Example 1

### Assay for PA-IL Antagonist Activity

Wells of a microtiter plate (plate 1) are coated with PA-IL (Sigma-Aldrich, St. Louis, Mo.) by incubation for 2 hrs at 37° C. The wells are then blocked for 2 hrs by the addition of 1% bovine serum albumin (BSA) diluted in TBS-Ca (50 mM TrisHCl, 150 mM NaCl, 2 mM CaCl₂ pH 7.4) mixed 1:1 v/v with Stabilcoat (Surmodics, Eden Prairie, Minn.). In a second low-binding round-bottom microtiter plate (plate 2), test antagonists are serial diluted in 1% BSA in TBS-Ca/Stabilcoat (60 μl/well). Preformed conjugates of α-galactose-PAA-biotin (GlycoTech Corp, Gaithersburg, Md.) mixed with streptavidin-HRP (KPL Labs, Gaithersburg, Md.) are added to each well of plate 2 (60 μl/well of 2 μg/ml). Plate 1 is then washed with TBS-Ca and 100 μl/well are transferred from plate 2 to plate 1. After incubation at room temperature for 2 hrs, plate 1 is washed and 100 μl of TMB reagent (KPL Labs, Gaithersburg, Md.) is added to each well. After incubation for 5 minutes at room temperature, the reaction is stopped by adding 100 μl/well of 1M H₃PO₄ and the absorbance of light at 450 nm is determined by a microtiter plate reader.

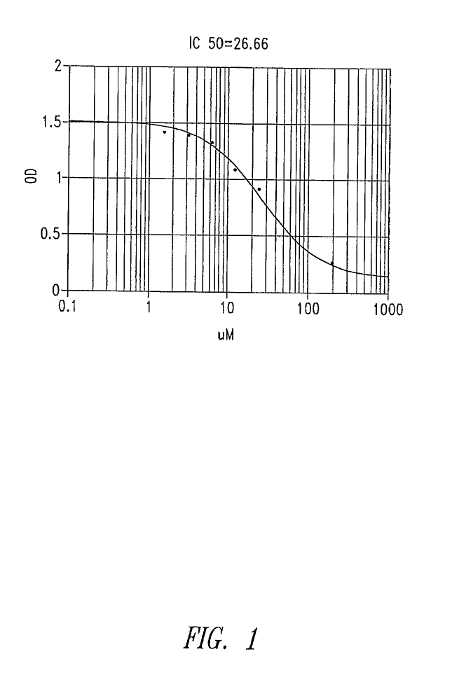

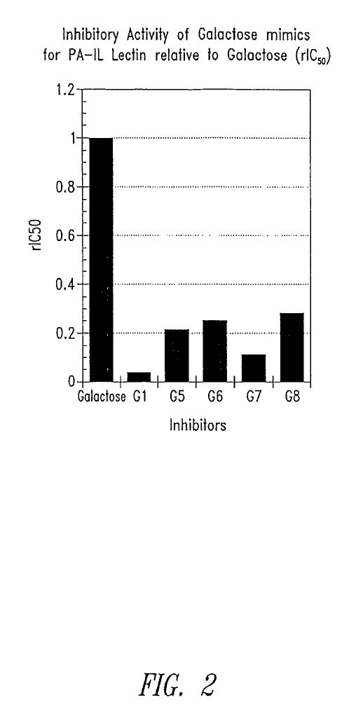

The results of use of the above assay with galactose or galactose mimics is shown in FIGS. 1, 2 and 3. The galactose mimics tested in FIG. 2 are depicted below:

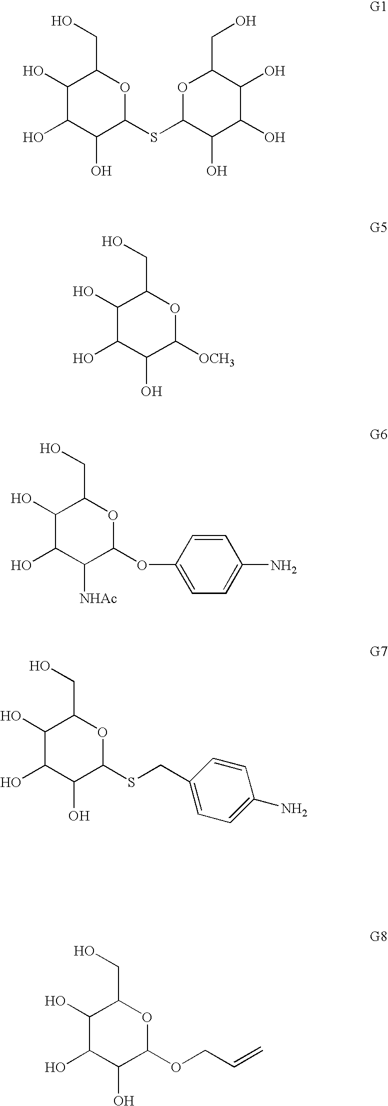

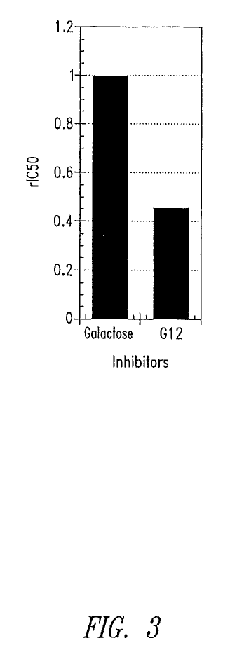

The galactose mimic tested in FIG. 3 is depicted below:

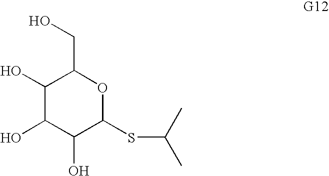

G1 was purchased from Sigma Chemical Co. (St. Louis, Mo.); and G5, G6, G7, G8 and G12 were purchased from Toronto Research Chemicals, Inc. (North York, Ontario, Canada).

### Example 2

### Assay for Inhibition of PA-I Lectin Binding to Buccal Cells

Obtain sample of buccal cells by scraping inside of cheek and collecting in 2 mls PBS. Spin cells at 400 g for 7 minutes to generate cell pellet. Discard supernatant. Resuspend in cold TBS-Ca (50 mM TrisHCl, 150 mM NaCl, 2 mM CaCl₂ pH 7.4) to cell concentration of 10⁶ cells/ml. Aliquot 0.1 ml to each tube. Add biotinylated PA-I to tubes (5 μ/well of 1.0 mg/ml lectin). Add inhibitors to tubes (5 μl at desired concentration). Incubate on ice for 30 minutes. Wash cells once by adding 400 μl of cold TBS-Ca to each tube and spinning at 400 g for 7 minutes. Discard supernatant. Resuspend cells in 100 μl of cold TBS-Ca. Adccd streptavidin-FITC (2 μl/tube of 1 mg/ml, KPL Labs, Gaithersburg, Md.). Incubate 30 minutes on ice. Wash cells once by adding 400 μl of cold TBS-Ca to each tube and spinning at 400 g for 7 minutes. Discard supernatant. Resuspend cells in 500 μl of cold TBS-Ca. Analyze in flow cytometer.

From the foregoing it will be appreciated that, although specific embodiments of the invention have been described herein for purposes of illustration, various modifications may be made without deviating from the spirit and scope of the invention.

1. A pharmaceutical composition comprising a pharmaceutically acceptable carrier or diluent in combination with a compound or physiologically acceptable salt thereof, said compound with the formula: 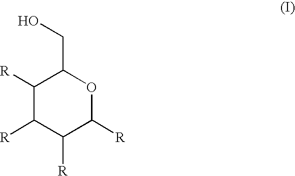
wherein each R is independently selected from OH, NHAc, alkyl, O-alkyl, S-alkyl, cycloalkyl, O-cycloalkyl, S-cycloalkyl, heterocycle, O-heterocycle, S-heterocycle, aryl, O-aryl, S-aryl, heteroaryl, O-heteroaryl, S-heteroaryl, and 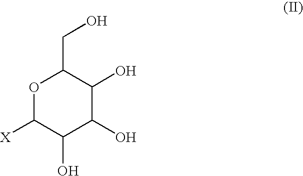
where X is S, O or CH₂, and with the bond of an R to the X of formula (II) where an R is selected from formula (II); and
with the proviso that the R of formula (I) are not all OH.

2. A composition comprising a compound or physiologically acceptable salt thereof, said compound with the formula: 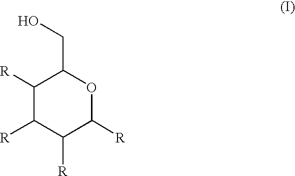
wherein each R is independently selected from OH, NHAc, alkyl, O-alkyl, S-alkyl, cycloalkyl, O-cycloalkyl, S-cycloalkyl, heterocycle, O-heterocycle, S-heterocycle, aryl, O-aryl, S-aryl, heteroaryl, O-heteroaryl, S-heteroaryl, and 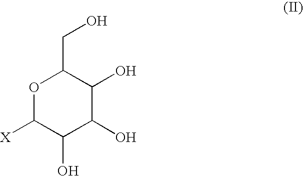
where X is S, O or CH₂, and with the bond of an R to the X of formula (II) where an R is selected from formula (II);
with the proviso that the R of formula (I) are not all OH; and
in combination with another inhibitor of Pseudomonas bacteria.

3. The composition of claim 2 further including a pharmaceutically acceptable carrier or diluent.

4. The composition of claim 1 wherein three of the R are OH in said compound.

5. The composition of claim 2 wherein three of the R are OH in said compound.

6. The composition of claim 3 wherein three of the R are OH in said compound.

7. The composition of claim 1, said compound with the formula: 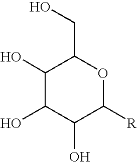
where R is as defined in claim 1.

8. The composition of claim 2, said compound with the formula: 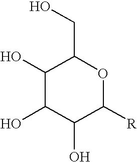
where R is as defined in claim 2.

9. The composition of claim 3, said compound with the formula: 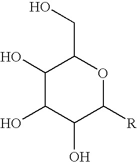
where R is as defined in claim 2.

10. The composition of claim 1, said compound with the formula:

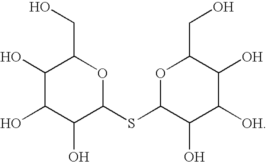

11. The composition of claim 2, said compound with the formula:

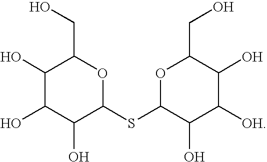

12. The composition of claim 3, said compound with the formula:

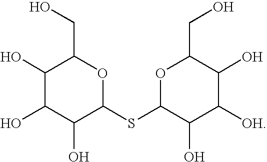

13. The composition of claim 1, said compound with the formula:

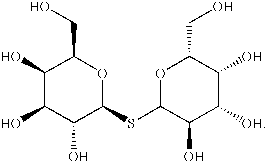

14. The composition of claim 2, said compound with the formula:

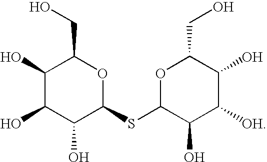

15. The composition of claim 3, said compound with the formula:

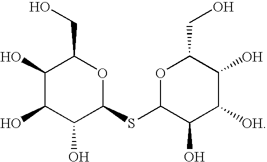

16. The composition of claim 1, further including a therapeutic agent for Pseudomonas bacteria therapy.

17. The composition of claim 1 wherein said compound is attached to a therapeutic agent for Pseudomonas bacteria therapy.

18. A conjugate comprising a compound or physiologically acceptable salt thereof joined covalently to a therapeutic agent for Pseudomonas bacteria therapy, said compound with the formula: 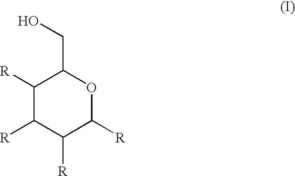
wherein each R is independently selected from OH, NHAc, alkyl, O-alkyl, S-alkyl, cycloalkyl, O-cycloalkyl, S-cycloalkyl, heterocycle, O-heterocycle, S-heterocycle, aryl, O-aryl, S-aryl, heteroaryl, O-heteroaryl, S-heteroaryl, and 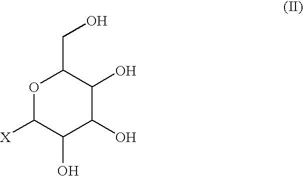
where X is S, O or CH₂, and with the bond of an R to the X of formula (II) where an R is selected from formula (II); and
with the proviso that the R of formula (I) are not all OH.

19. The conjugate of claim 18 wherein three of the R are OH in said compound.

20. The conjugate of claim 18, said compound with the formula: 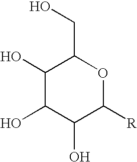
where R is as defined in claim 18.

21. The conjugate of claim 18, said compound with the formula:

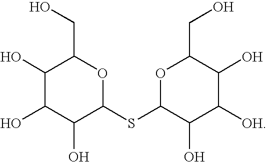

22. The conjugate of claim 18, said compound with the formula:

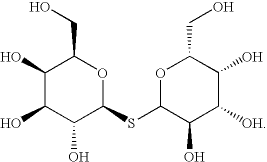

23. A method of inhibiting Pseudomonas bacteria infection in a warm-blooded animal comprising administering to the animal in an amount effective to inhibit PA-IL lectin of the bacteria a composition comprising pharmaceutically acceptable carrier or diluent in combination with a compound or physiologically acceptable salt thereof, said compound with the formula: 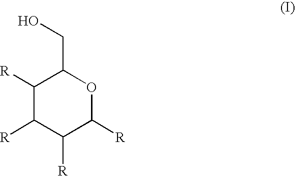
wherein each R is independently selected from OH, NHAc, alkyl, O-alkyl, S-alkyl, cycloalkyl, O-cycloalkyl, S-cycloalkyl, heterocycle, O-heterocycle, S-heterocycle, aryl, O-aryl, S-aryl, heteroaryl, O-heteroaryl, S-heteroaryl, and 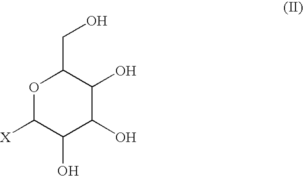
where X is S, O or CH₂, and with the bond of an R to the X of formula (II) where an R is selected from formula (II); and
with the proviso that the R of formula (I) are not all OH.

24. The method of claim 23 wherein the composition further includes a therapeutic agent for Pseudomonas bacteria therapy.

25. The method of claim 23 wherein said compound of the composition is attached to a therapeutic agent for Pseudomonas bacteria therapy.

26. The method of claim 23 wherein the bacteria are Pseudomonas aeruginosa.

27. A method of detecting Pseudomonas bacteria comprising contacting a sample with a diagnostic agent linked to a compound, under conditions sufficient for the compound to bind to the bacteria or PA-IL lectin product if present in the sample; and detecting the agent present in the sample, wherein the presence of agent in the sample is indicative of the presence of Pseudomonas bacteria; said compound with the formula: 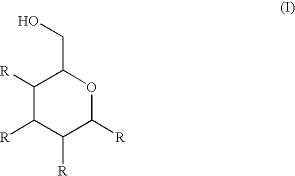
wherein each R is independently selected from OH, NHAc, alkyl, O-alkyl, S-alkyl, cycloalkyl, O-cycloalkyl, S-cycloalkyl, heterocycle, O-heterocycle, S-heterocycle, aryl, O-aryl, S-aryl, heteroaryl, O-heteroaryl, S-heteroaryl, and 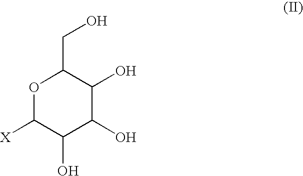
where X is S, O or CH₂, and with the bond of an R to the X of formula (II) where an R is selected from formula (II); and
with the proviso that the R of formula (I) are not all OH.

28. The method of claim 27 wherein three of the R are OH in said compound.

29. The method of claim 27, said compound with the formula: 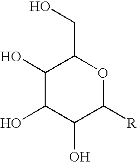
where R is as defined in claim 27.

30. The method of claim 27, said compound with the formula:

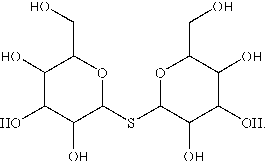

31. The method of claim 27, said compound with the formula:

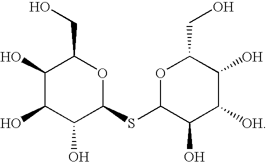

32. The method of claim 27 wherein the bacteria are Pseudomonas aeruginosa.

33. A method of immobilizing Pseudomonas bacteria on a solid support comprising contacting, under conditions sufficient for binding, a sample containing Pseudomonas bacteria with a compound that is immobilized on a solid support; and separating the sample from the solid support; said compound with the formula: 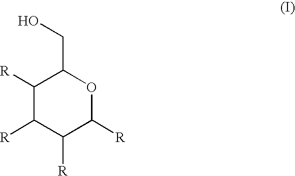
wherein each R is independently selected from OH, NHAc, alkyl, O-alkyl, S-alkyl, cycloalkyl, O-cycloalkyl, S-cycloalkyl, heterocycle, O-heterocycle, S-heterocycle, aryl, O-aryl, S-aryl, heteroaryl, O-heteroaryl, S-heteroaryl, and 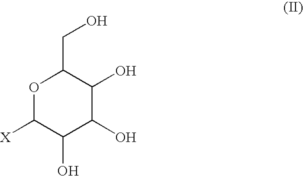
where X is S, O or CH₂, and with the bond of an R to the X of formula (II) where an R is selected from formula (II); and
with the proviso that the R of formula (I) are not all OH.

34. The method of claim 33 wherein three of the R are OH in said compound.

35. The method of claim 33, said compound with the formula: 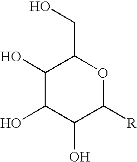
where R is as defined in claim 33.

36. The method of claim 33, said compound with the formula:

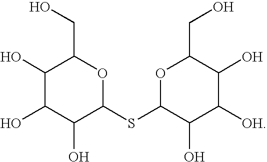

37. The method of claim 33, said compound with the formula:

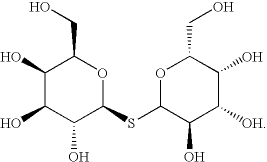

38. The method of claim 33 wherein the bacteria are Pseudomonas aeruginosa.

39.-40. (canceled)
